(19) United States
(12) Patent Application Publication
(10) Pub. No.: US 2026/0183313 A2
(43) Pub. Date: Jul. 2, 2026

(54) Post COV19 Vaccine Remedies

(71) Applicant: Suzanne J. Paxton-Pierson, Poulsbo, WA (US)

(72) Inventor: Suzanne J. Paxton-Pierson, Ellensburg, WA (US)

(21) Appl. No.: 17/482,364

(22) Filed: Sep. 22, 2021

(51) Int. Cl. A61K 31/593; A61K 31/19; A61K 31/353; A61K 31/375; A61K 45/06

(52) U.S. Cl. CPC A61K 31/593; A61K 31/19; A61K 31/352; A61K 31/353; A61K 31/375; A61K 45/06

(57) ABSTRACT

Methods to lessen toxicities in COV19 vaccine aftermath.

Hereinafter, the COVID-19 vaccine, CV19 vaccine, injections, or exposure shall be referred to as “CVx”.

Disclaimer: Nothing herein is medical advice, a prescription, recommendation, or treatment guidance. Certain items are prophetic, speculative and for academic research only. Where prophetic examples are presented, they describe reasonably expected or anticipated results based on known principles and are not based on actual experiments performed. DO NOT replicate, synthesize, test, administer, or use any content in this specification. Consult a qualified healthcare professional for any health concerns. NO WARRANTIES of safety, efficacy, or suitability. Examples illustrate structural/mechanistic concepts only. Certain referenced compounds are known to be toxic and are not intended or recommended for any use, experimentation, or application. This disclosure does not enable or suggest harmful or unauthorized uses.

There are urgent interventions needed to prevent untoward adverse genetic and toxic events and death in certain persons receiving the CVx. Many aspects of the CVx, its full complement of ingredients, binary interplay with EMF/RF, and toxicity etiology, at this time, remain unknown, tentatively known, or unclear.

On Oct. 22, 2020 CBER FDA published adverse event and toxicities from the CVx including: Death; Guillain-Barre Syndrome; Acute Disseminated Encephalomyelitis; Transverse Myelitis (polio symptoms); Encephalitis; Myelitis: Encephalomyelitis; Meningoencephalitis; Meningitis; Encephalopathy; Convulsions; Seizures; Stroke; Narcolepsy; Cataplexy; Anaphylaxis, Acute Myocardial Infarction; Myocarditis; Pericarditis; Autoimmune Diseases; Miscarriages; Stillbirths; Demyelinating Diseases; Non-Anaphylactic Allergic Reactions; Thrombocytopenia; Disseminated Intravascular Coagulation; Venous Thromboembolism; Arthritis; Arthralgia; Joint Pain; Kawasaki Disease; Multisystem Inflammatory Syndrome in Children (MIS-C); Vaccine Enhanced Disease; Antibody Dependent Enhancement; Pathologic Priming] as well as Neurological Diseases including Spongiform Encephalopathy, Early Onset Dementia and Alzheimer's, Solid Tumors, Blood Cancers. Many others resultant diseases are as yet to be discovered and no long-term safety data exist. Post CVx symptoms may be caused by singular ingredients or various ingredients or in conjunction with external radiation sources which may cause synergistic toxicities. Many ingredients may not be listed on a legal, official package insert as yet. These ingredients may at least include: mRNA or adenovector routes to production of the synthetic S1 antigen spike protein, metals.

Prevention of mRNA translation via destruction or disabling of the mRNA or adenovector DNA; the amount of toxic spike protein production may be inhibited or stopped. S1 spike protein shedding may also cause “occupational secondary adverse events” to persons in proximity or exposed to bodily fluids. mRNA and adenovector DNA are sensitive to heat, therefore immediate local heat treatments within about 8 inches of the injection, particularly above about 37 C when employed, may denature the genetic payload. detergents violate the lipid micelle structures, exposing the genetic payload for reduction. Binding of cationic lipid nanoparticles to prevent DNA binding and damages with phospholipids (negatively charged) lecithin, which contains choline phosphate plus glycerol, may reduce cell entry.

Protease inhibitor-reverse transcriptase inhibitors may be employed. Halting DNA synthesis temporarily can prevent DNA alterations by intercalation of adenovector payloads. Protection of DNA from mRNA reverse transcription and vector chromosomal integration may be facilitated by halting DNA synthesis temporarily in order to prevent DNA alterations from adenovector payload or retroviral material which may be reverse-transcribed by intrinsic reverse transcritpases. Proteolytic enzymes such as bromelain and papain may inhibit or stop, temporarily, DNA synthesis, which restarts in 48 hours at an accelerated rate. This method is exploitable for preemptive radiotherapy radioprotection as well. Pineapple's bromelain is a proteolytic enzyme extract from pineapple stems.

Aminothiols alter chromatin structure and also inhibit DNA synthesis, causing observed post-treatment inhibition of DNA formation and subsequent blockage of cells in late S or G2 phases. Polyamine depletion post-gene therapy may improve DNA repair fidelity. Polyamine depletion stalls DNA synthesis (70-80%) as shown by DFMO-treated cells in which DFMO inhibits ODC, the first enzyme in polyamine biosynthesis. Cysteamine inhibits DNA synthesis in mouse bone marrow. WR-1065 can distort DNA supercoiling and this perturbation could stall DNA synthesis allowing for less intercalation. WR-2721 has close structural similarity to the polyamines spermine and spermidine, which implicates its effects on DNA synthesis, DNA repair and genomic stability. WR-2721 and its metabolites compete with putrescine for uptake into rat lung slices via the polyamine transport system. Both polyamines and aminothiols stabilize DNA and affect chromatin structure and condensation. Aminothiols may also bind and stabilize DNA which is not covered by histones. WR-1065 can distort DNA supercoiling and this perturbation could stall DNA synthesis.

Thymoquinone and other Nigella sativa seed constituents through inhibition of cell growth in G1 phase allow inhibition of DNA synthesis. Nigella sativa oil injected in mice protects normal tissues and prevent spike protein damages. Zinc is a reverse transcriptase inhibitor and quercetin and hydroxychloroquine or chloroquine serve as ionophores for zinc. Diallyl sulfide inhibits nuclear aberrations possibly from mRNA or pDNA; its radioprotection effects are dose-dependent but must be dosed prior. DAS will suppress DNA synthesis and ODI activity (which regulates DNA synthesis); both DNA synthesis and ODI activity are normally elevated after radiation or damages/alterations. DFMO abolished DAS activity to radio-protect against colonic nuclear damage; thus DAS radioproprotects via a polyamine-related pathway. Dry fasting and electrolyte fasting reduce DNA alterations and spike protein manufacture.

The S1 antigen spike protein ACE2 receptor binding domain causes destruction of ACE2 receptors. ACE inhibition results in lower angiotensin II. ACE2 is inhibited by ACE2 residues; Q24, D30, H34, Y41, Q42, M82, K353 and R357; a high lysine diet with heightened arginine may reduce ACE2 affinity and interferes with fusion domain for S1 spike protein by 10%; a more alkaline blood may dimish as achieved by sodium bicarbonate. L-lysine blocks L-arginine transport in vitro, via competitive blockade and down-regulation of cationic amino acid transporters. Dose of up to 3 g/day lysine very safe, up to 10-15 g/day (which may result in diarrhea). ACE2 destruction may lead to mitochondrial ablation, causing CHF, stroke, and pulmonary hypertension. Suramin, and pine needle extract may limit mitochondrial destruction. Increased G and C in the mRNA increases protein. Ivermectin may block ACE2-receptors. Angiotensin receptor protect from spike protein destruction, which may result in greater survival. A reduction of mortality may result from using ACE-inhibitors/ARBs or means to upregulate ACE2 in CV. ACE-I and ARBS in a CVx patient may upregulate ACE2 enzyme which is protective. Blocking of ACE2 receptors from spike protein damages may help preserve health. The S1 spike protein pharmacokinetic data show that it is produced ubiquitously, with uptake into spleen, bone marrow, ovaries, testes, lungs and brain. The S1 antigen alone may cause all COVID19 symptoms. L-carnosine, vitamin D3, melatonin may reduce spike protein ACE2 toxicity; dry fasting and electrolyte fasting may reduce spike protein production.

mRNA when used for cancer treatments activated oncogenes and tumor suppressor genes turned off, and CVx may cause lowered p53 apoptosis. Dry fasting or electrolyte fasting, sauna use, enhance heat shock proteins and p53 apoptosis and autophagy; HCQ and fenbendazole have anticancer properties. Heavy metal analyses have shown the presence of chromium, barium, tungsten, and iron. EDTA and sodium ferrocyanide can assist in clearing heavy metals which may cause oxidative and other damages. Sodium ferrocyanide has shown a similarly low oral toxicity with an LD50 in rat at 980 mg/kg (Chemical Abstract Service data for sodium ferrocyanide). Sodium ferrocyanide USP is a potentially more widely available, higher-affinity extreme-low toxicity orally-dosable alternative chelation agent compared to others.

Inflammatory cytokines, higher phospholipase A2 and ROS commensurate with the CVx will be aided by curcumin/turmeric, C60, vitamin C, other antioxidants, and honey. Guillain Barre is linked to inflammatory cytokines. The tat protein calms GBS and turmeric duplicates the effect. Queensland University CVx patients tested HIV-positive: GBS can be the first sign a patient is HIV-positive. Guillain-Barré Syndrome (GBS) is an acute demyelinating polyneuropathy which can occur post-infection. Several viral infections have been associated with GBS including cytomegalovirus (CMV), Hepatitis E virus (HEV) and Zika virus. Triumeq, a once daily tablet combination of the antiretrovirals abacavir, lamivudine and dolutegravir has proven useful in HIV+ GBS patients. CMV is a latent herpes DNA virus with retroviral components which causes retinal blindness. EBV is a latent herpes DNA cancer virus which awakens dormant HERV-W, a retrovirus.

Many naturally derived anti-HIV compounds are flavonoids, coumarins, terpenoids, alkaloids, polyphenols, polysaccharides or proteins. Natural product neurologically-active gp41 fusion inhibitors include theaflavin, theaflavin-3-gallate, theaflavin digallate, epitheaflavin-3V-gallate, gallocatechin gallate, epigallocatechin gallate EGCG, epigallocatechin 3,4 digallate and 2,2-bisepigallocatechin digallate. Cistus incanus extract and cinnamon-derived procyanidin compound type A polyphenol trimer, particularly A-type procyanidin oligomers, displays anti-HIV-1 as a gp120 inhibitor for co-receptor-binding sites on gp120. An extract from Spirulina, has documented antioxidant, immunomodulatory, anti-inflammatory and antiviral activities. Other RTase anti-HIV compounds are anti HIV alstonine-HCl, arctigenin, baicalein, berberine, columbamine, digallic acid, ellagic acid, jatrorrhizine, kaempferol, punicacortein, punicalin, quertagetin, quercetin, and trachelogenin.

Resultant immunodeficiencies post CVx such as CD8 and comprehensive antibodies may be aided by vitamin D3, 25-OH vitamin D and calcifediol (soluble in DMSO and ethanol) which have been associated with lower mortality. Honey, ashwaghanda and mushrooms can improve immunity. Lentinan (Shiitake) compounds are antiviral, immunostimulant and antitumor. Whole Shiitake mushroom may be used as food or taken as tea. Lentinan (1,3 β-D-glucan) is a polysaccharide isolated from shiitake mushrooms which has anti-cancer effects in colon cancer and leukemia cells. Lentinan has anti-fungal and anti-HIV-1 reverse transcriptase activity. Lentinan has anti-fungal and anti-HIV-1 reverse transcriptase activity. Lentinan is antiproliferative, immunostimulatory, hepatoprotective, antimutagenic, and increases CD4 and neutrophil activity. Shiitake improves quality of life and survival in various cancer patients. Mushroom extracts are pro-immunity such as Ganoderma lucidum (Reishi), Lentinus edodes (Shiitake), Inonotus obluquus (Chaga), Maitake. Shiitake compounds are antiviral, immunostimulant and antitumor. Chaga increases tolerance to radiation. Chaga mushroom reduced toxicity associated with radiation in animal models. IVMN tells functional adapative T cells how to act. Antibody dependent enhancement (ADE) or pathogenic priming results from spike protein expression on the cell surface and autoimmune attack upon re-exposure to wild type virus, along with overall comprehensive antibody titer flattening with subsequent boosters. Improvement of natural immunity to restore reactivity will be preferential to boosters.

In treatment for coronaviruses, hydroxychloroquine, chloroquine, ivermectin, with dexamethasone or budesonide and other anti-inflammatories such as turmeric/curcumin or NSAIDS such as naproxen, aspirin, ibuprofen, etc. are possibly of some benefit for cytokine storm. The spike protein surface expression alters capillary endothelium interaction with platelets which interpret the roughness as damages and react in coagulation cascades particularly with damaged RBC. Red blood cell morphology changes with CVx, either destroying cells, causing stacking, or clumping. Hydration, exercise, anticoagulants are important to preventing clots. D-dimer and fibrinogen tests for microclots steer treatments to occult nascent tissue damages or tissue death; troponin tests can measure cardiac and skeletal muscle death. Treatment with resveratrol and vitamin B3 may replace some destroyed cells with adult stem cells.

Destruction of mitochondria leads to lethargy, CHF, lack of vitality. Nicotinamide mononucleotide is a precursor to NAD+, useful to gain more cell energy; caffeoylquinic acid, vitamin D3 with C60 reduces ROS can help mitochondrial health to produce normal ATP.

If the synthetic spike protein syncytin homology is expressed, this is pro-cancer. IVMN and licorice are antisyncytia compounds. 18β-GA inhibits viral attachment, internalization, and by stimulating IFN secretion. Ingestion of spirulina or of spirulina extracts enriched in phycocyanobilin may have potential for boosting type 1 interferon response in the context of RNA virus infection.

Zinc is able to up-regulate host immune response antiprotozoal activity Hydroxychloroquine, ivermectin, fenbendazole, oxytetracycline, doxycycline, amoxicillin, clindamycin, zinc 25 mg, sodium bicarbonate and borax.

Prions are replicated, misfolded proteins in the spike protein which disrupt the FUS gene causing ALS or cancer. The Frontal Temperal Lobe FTD43 protein FTL degenerates and causes early Alzheimer's and dementia and 5 kinds of neurological diseases whereby the patient loses speech and facial recognition. EOD is seen in ages 45-50 called early onset dementia. Epigallocatechin gallate and heat shock protein (which spikes with dry fasting and saunas) may limit dementia and Alzheimer's.

The synthetic PEG technology of fatty lipoid liquid nano-crystals as carriers which encase genetic payload forms a sturdy LDL-like pseudovirus lipid nanoparticle. PEG poses an anaphylaxis risk and lung toxicity risks. It is prudent to have epinephrine, norepinephrine, noradrenaline, and beta agonists on hand.

While the spike protein attaches to ACE2 receptor, based on the clinical observation of lower prevalence of smoking in hospitalized CV19 patients, the RBD of the S1 spike protein may bind to nicotinic acetylcholine receptors. There is a toxin-like amino acid sequence in the receptor binding domain of the S1 spike protein (aa 375-390) homologous to neurotoxic homolog NL1 (which is a snake venom toxin known to interact with nicotinic acetylcholine receptors (nAChRs) whereby nicotine and other nicotinic cholinergic agonists such as NAChR neuronal agonists such as cytisine of Fabaceae family, may protect nAChRs and thus have therapeutic value in CV19 vaccine injection patients.

nanoparticles, undisclosed adjuvants; Purging may be possible via chelating agents including sodium ferrocyanide, EDTA, magnetic forces, nicotine, ethanol, vitamin C, glutathione or glutathione precursors N-acetyl cysteine, selenomethionine, sodium selenite, C60 antioxidant, and turmeric.

Other possibly useful compounds, ursolic acid, betulinic acid, betulin, chlorogenic acid, caffeic acid, uvaol, ursolic acid, alpha-amyrin, beta-sitosterol, campestrol, choline, diacetyl-nerigoside, quercetin, quercitrin, rosaginin, rutin, stearic acid, stigmasterol, oxyresveratrol, tangeretin, nobiletin, glycyrrhizic acid, quercetin 3 rhamnoside, samarangenin, tetranortriterpenoid 1-cinnamoyl-2, 11-dihydroxymeliacarpin, pterocarnin A, chalepin, pseudane IX, manassantin B, dicaffeoylquinic acids, scopadulcic acid, trihydroxy-8-methoxyflavone, baicalin, glycyrrhiza chalcones, dammarenolic acid, ajoene, allicin, allyl methyl thiosulfinate, methyl allyl thiosulfinate, anthraquinone aloe emodin, triterpene saponin, and 13-deoxy-11,12-didehydroandrographolide, sulfated polysaccharides, quercetin derivatives, lectins of Canavialia ensiformis, anthraquinones of Cassia angustifolia, acidic polysaccharides of Cedrela tubiflora, phenolic compounds of Cicer arietinum, catechin, epicatechin and B-type procyanidins of Cocoa nucifera, alkenes and amides of Echinacea purpurea, polysacchardic, choric acid and others of Echinacea purporea, sesquiterpene sclerocarpic acid of Glyptopetalum sclerocarpum, oligomeric to polymeric proanthocyanidins.

Other antihistamines: loratadine, hydroxyzine, carbinoxamine, cryproheptadine, desloratidine, livostin, levocetirizine, brompherinamine, ceterizine, chlorpheniramine, clemasatine, diphenhydramine, fexofenadine.

Other steroid type compounds: budesonide, dexamethasone, medicaments including corticosteroids hydrocortisone, cortisone, betamethasone, prednisone, prednisolone, triamcinolone, methylprednisolone, fludrocortisone.

Other beta receptor type compounds: albuterol, formoterol, etc. salmeterol, vilanterol, levalbuterol, olodaterol.

Other adrenergic-type anti-anaphylaxis compounds: epinephrine, norepinephrine, pseudephedrine, phenylephrine,

Doses are in the therapeutic 0.0001% to 95% w/w or w/v range.

1. A method for mitigating negative effects of comprising administering to a human or mammal subject in need thereof, of a synergistic, therapeutically-effective amount of 18-beta Glycyrrhetinic Acid, Epigallocatechin Gallate, Spirulina extract, Cinnamon extract, Curcumin, Betulinic Acid, Vitamin D3, Zinc Ascorbate, Hesperitin, Luteolin, Sodium Ferrocyanide, nicotinamide mononucleotide.

2. The method of claim 1 with the option of at least one or more of the following physical or drug treatments for CVx:
a—one or more in a composition of histamine type anti-anaphylaxis compounds including anti-IgE, antihistamine medicaments including loratadine, hydroxyzine, etc. and
b—one or more in a composition of steroid type anti-anaphylaxis compounds including budesonide, dexamethasone, etc. and
c—one or more in a composition of beta receptor type anti-anaphylaxis compounds including beta receptor medicaments albuterol, formoterol, etc., and
d—one or more in a composition of adrenergic-type anti-anaphylaxis compounds including, epinephrine, etc., and
e—
f—
g—one or more in a composition of decorporation agents including activated charcoal, Attapulgite, Bentonite clay, calcium alginate, chlorella, ethylenediaminetetraacetic acid, French Green clay, Fuller's Earth, green tea extract, Montmorillonite clay, Pascalite, psyllium husk powder, citric acid, calcium citrate, and Zeolite, sodium silicate, potassium silicate, silica, silicon dioxide, and
h—application of high thermal temperature optimally within the first minutes or hours after CVx, of up to about 135° F. for up to about 8 seconds, up to 125° F. up to 1 minute and up to about 120° F. up to about 8 minutes, as well as lower temperatures above 105° F. for longer durations, local heat treatments directly upon and within about 8 inches of the injection, particularly above about 37° C., and
I—
j—subjecting the affected area or mammal to a strong field, pulsed or by alternating current demagnetization, or applying a reversed magnetic field and
k—detergents locally at the same or nearby site, and
l—administration of metal chelators comprised of sodium ferrocyanide, or EDTA administered by slow IV infusion, and therapeutically-effective amounts of mint, polyphenols, flavonoids, rose hips, alpha-lipoic acid, N-acetyl cysteine, and
m—ashwagandha, metformin and
n—one or more in a composition of anti-anaphylaxis, anti-inflammatory, anti-cytokine, antiprostaglandin medicants of benefit for cytokine storm including NSAIDS such as naproxen, acetyl salicylic acid, glabridin, ibuprofen, meclofenamate sodium, diflunisal, tolmetin, ketoprofen, flurbiprofen, ketorolac, tromethamine, indomethacin, fenoprofen, diclofenac, celecoxib, omega-three fatty acids, or hydroxychloroquine, chloroquine, ivermectin, and turmeric or curcumin, and
o—one or more in a composition of antiretroviral protease inhibitor medicaments abacavir, lamivudine and dolutegravir, atazanavir, darunavir, fosamprenavir, indinavir, lopinavir, ritonavir, nelfinavir, pentamidine (aerosolized or not), zidovudine; glycyrrhizin, glycyrrhetinic acid, betulinic acid, quercetin, salicin, camostat mesylate, and silybin and antisyncytium glycyrrhetinic acid, ivermectin, and berberine, and
p—one or more in a composition of a composition of possible anti-spike protein medicaments including hydroxy-chloroquine, chloroquine, quinine, artemisinin, Artemisia, fenbendazole, ivermectin, suramin, pine-needle extract, azithromycin, quercetin, vitamin D3, zinc, vitamin C, an alkaline high lysine diet with heightened arginine intake, sodium bicarbonate, melatonin, dry fasting, electrolyte fasting, and sauna use to reduce spike protein production, and
q—
r—administration of negatively-charged binder of cationic lipid nanoparticles lecithin, and
s—one or more in a composition of DNA synthesis halting agents proteolytic enzymes bromelain and papain, DFMO, aminothiols amifostine, WR-2721, WR-1065, cysteamine, thymoquinone and other Nigella sativa extracts, and diallyl sulfide DAS and
t—one or more in a composition of one or more of a composition of antioxidants and omega three medicaments including C60, curcumin/turmeric, omega-three fatty acids, vitamin E, vitamin C, selenium, SOD, glutathione, flavonoids; flavonols; flavones; catechins; flavanones; polyphenols, anthocyanidins; isoflavonoids; and/or plant, vegetable, or fruit extracts including those obtained from green tree leaves, milk thistle, soybeans, wine grapes and their seeds, acai berry, coffee berry, feverfew, pomegranate, tropical ferns, turmeric, and witch hazel, EGCG, SOD, glutathione, vitamin D3, melatonin, GSH, ALA, beta carotene, catalase, GST, CoQ10, mint rose hips, vitamin A, famotidine, etc., N-acetyl cysteine, selenomethionine, sodium selenite, and
u—one or more in a composition of one or more of a composition of ACE-II receptor protection medicaments: kaemperfol, apigenin, quercetin, luteolin, emodin, chrysin, rhein, delphinidin, cyanidin, apigenin, rhoiflolin, rutin, quercetine, nicotianamine, catechin, epigallocatechin, epigallocatechin gallate, ferlic acid, chlorogenic acid, isoferulic acid, caffeic acid, delta-viniferin, myritilin, myricitrin, lactucopicrin 15-oxalate, nympholide afzelin, biorobin, phyllaemblicin B, baicalin, hesperitin, scutellarin, glycyrrhizin, curcumin, tangeretin, nobiletin, naringenin, brazilein, brazilin, galangin, and acetoxychavicol acetate, captopril, analapril, ramipril, trandolapril, perindopril, benazepril, fosinopril, temocapril, L-carnosine, silybin, and salicyl-carnisine, losartan, irbesartan, valsartan, azilsartan, candesartan, eprosartan, olmesartan, telmisartan, high lysine, high arginine, sodium bicarbonate, and
V—one or more in a composition of one or more of a composition anticoagulant compounds as needed including therapeutic amounts of water warfarin, coumarin, heparin, apixaban, dabigatran, edoxaban, enoxaparin, allicin, ajoene, and S-allyl cysteine, hydration, increased exercise, NSAIDS, and
W—one or more in a composition of one or more of a composition antiprotozoal compounds (and for associated infections) chloroquine, ivermectin, hydroxychloroquine and fenbendazole, pentamidine (aerosolized or not), zidovudine; benznidazole and also nifurtimox, ivermectin, oxytetracycline, doxycycline, amoxicillin, clindamycin, doxycycline, amoxicillin or cefuroxime, zinc, sodium bicarbonate, and
x—one or more in a composition of one or more of a composition immune enhancement compounds vitamin D3, and honey, mushroom extracts Ganoderma lucidum (Reishi), Lentinus edodes (Shiitake), Inonotus obluquus (Chaga), Maitake, Glycyrrhizae pro-interferon 18β-Glycyrrhetinic Acid, interferon, and
y—one or more in a composition of one or more of a composition anti-HIV compound: gp41 fusion inhibitors theaflavin, theaflavin-3-gallate, theaflavin digallate, epitheaflavin-3V-gallate, gallocatechin gallate, epigallocateching gallate EGCG, epigallocatechin 3,4 digallate, 2,2-bisepigallocatechin digallate and gp120 inhibitor Cistus incanus extract, cinnamon-derived procyanidin compound type A polyphenol trimer, A-type procyanidin oligomers, and antiviral extract from Spirulina, an antioxidant, immunomodulatory, anti-inflammatory and antiviral substance, and
Z—one or more in a composition of antineurotoxicity and anti-GBS medicaments NAChR cholinergic neuronal agonists nicotine, cytisine, anabseine, C60, curcumin, turmeric, and
aa—one or more in a composition of a composition pro-mitochondrial medicaments nicotinamide mononucleotide a precursor to NAD+, NMN, vitamin D3, caffeoylquinic acid or cinnamic acid, C60 to reduce ROS, and resveratrol.
